(19) United States
(12) Patent Application Publication
(10) Pub. No.: US 2004/0208943 A1
(43) Pub. Date: Oct. 21, 2004

(54) Method for treatment of skin disorders

(76) Inventor: Bronhilda Miketin, Duluth, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/419,517

(22) Filed: Apr. 21, 2003

(51) Int. Cl. A61K 35/78

(52) U.S. Cl. 424/730; 424/764; 424/774

(57) ABSTRACT

This invention relates to the natural topical treatment of portions of skin of a person afflicted with a skin disorder such as dry skin, eczema, itchy skin, red skin, itchy eczema, inflamed skin, and/or cracked skin for the removal of itch and the restoration of the affected areas of skin to a normal condition. The natural treatment of a skin disorder initially involves formation of a natural ointment from the ingredients identified as lard; marigold flowers; pure bee's wax; incense; rosemary flower; nettles extract; marigold oil; and St. Johns wart oil. The natural ointment is formed by combination of the ingredients which includes heating. The ointment is then applied twice daily to affected areas of skin until a natural cure of the skin disorder is obtained.

### STATEMENT REGARDING FEDERALLY SPONSORED RESEARCH OR DEVELOPMENT

(Not Applicable)

### RELATED CASES

(Not Applicable)

### BACKGROUND OF THE INVENTION

The present invention relates to a topical composition for the treatment of inflammation, rashes, hives, burns, psoriasis, hemorrhoids, poison ivy, insect stings, cuts, aging skin, and other types of undesirable skin disorders including but not necessarily limited to dry skin, eczema, itchy skin, red skin, itchy eczema, inflamed skin, and/or cracked skin. Skin disorders may generally be evidenced by the presence of skin elevations and scales which may be silvery in appearance. Psoriasis in general is a disease which accelerates the epidermal proliferation and proliferation of capillaries in the dermal region. In addition, psoriasis frequently results in the evasion of the dermis and epidermis by inflammation of the affected cells.

Areas of skin affected by psoriasis and other types of undesirable skin disorders also frequently lose water significantly faster than normal healthy skin. The areas of skin affected by skin disorders therefore tend to have increased metabolic rates which in turn causes a negative impact on tissue catabolism and potentially results in muscle wasting.

Skin disorders and Psoriasis as a chronic skin disease have been difficult to treat. Skin disorders may affect an individuals skin proximate to elbows, knees, trunk, and scalp. In the past, the treatment of skin disorders has included the use of various chemical agents such as dihydroxyanthralin, azarabine, colchicine, fluorouracil, methotrexate, methoxsalen, and the use of ultra-violet light. These methods have generally not provided satisfactory treatment of the skin disorder for individuals.

Alternatively, therapeutic regimes for the treatment of skin disorders include topical or intra-lesional application of corticosteroids, topical administration of anthralin or keratolytics, and the use of ultra-violet light on the affected areas. As is known in the art, no single therapy is ideal for the treatment of skin disorders and it is extremely rare for a patient to not receive treatment from several different therapeutic alternatives to attempt to prevent relapse and/or obtain remission for the skin disease. In addition, individuals frequently are required to be exposed to increased doses of medication which may magnify side effects adversely affecting the health of the individual.

Psoriasis is a relatively common skin disorder in which cell proliferation is increased up to 10 times the normal rate for an individual. The skin is the largest portion of the human body which is comprised of cells within three skin layers. Each of the skin layers is in a constant state of growth with the outer layer being formed of predominantly dead tissue which is naturally being discarded at a normal rate. Replacement of cells from underlying layers is accomplished by cell division and maturation where cells move upwardly and outwardly at a rate which varies dependent upon the age, sex, and/or health of an individual. Skin disorders commonly cause an increased rate of turn over of cells within an affected area which in turn increases the rate of cell growth and cell death. This increased rate of cell growth and cell death may result in injuries and/or disorders which accompany the increased synthesis of all tissue components and further elevate the strain placed upon skin or other tissue and the bio-synthetic capabilities of the cells within the affected area. The method and treatment of skin disorders as disclosed herein preferably slows cell proliferation and naturally treats areas of skin affected by one or more skin disorders. An individual's skin may then heal naturally, minimizing the risk of undesirable side effects and the relapse of the undesirable skin condition.

### BRIEF DESCRIPTION OF THE INVENTION

This invention relates to the natural topical treatment of portions of skin of a person afflicted with one or more types of skin disorders such as dry skin, eczema, itchy skin, red skin, itchy eczema, inflamed skin, and/or cracked skin for the removal of itch and the restoration of the affected areas of skin to a normal condition. The natural treatment of one or more types of skin disorders initially involves formation of a natural ointment from the ingredients identified as lard, marigold flowers, incense, rosemary flower, nettles extract, marigold oil, St. Johns wart oil, and pure beeswax. The natural ointment is formed by combination of the identified ingredients and the application of heat and stirring. The ointment is then applied twice daily to affected areas of skin until a natural cure of the undesirable skin condition is achieved.

It is a principal advantage of the present invention to provide a method for treatment of undesirable skin disorders formed of relatively simple and inexpensive components which is safe and which fulfills the intended purpose for alleviation of symptoms and the provision of a cure for an undesirable skin disease without fear of further injury to individuals.

Another principal advantage of the present invention is the provision of a method for treatment of undesirable skin disorders which includes a topical treatment completely formed of natural ingredients.

Still another principal advantage of the present invention is the provision of a method for treatment of skin disorders which reduces and/or eliminates “itchiness” of skin which is a common symptom/condition of a skin disorder.

Still another principal advantage of the present invention is the provision of a method for treatment of skin disorders which reduces and/or eliminates the undesirable “redness” or discoloration appearance of skin which is a common symptom of a skin condition.

Still another principal advantage of the present invention is the provision of a method for treatment of skin disorders which reduces and/or eliminates undesirable skin scales which is a common symptom/condition of a skin disorder.

Still another principal advantage of the present invention is the provision of a method for treatment of skin disorders which slows moisture loss of affected skin and tissue which is a common symptom/condition of skin disorders.

Still another principal advantage of the present invention is the provision of a method for treatment of skin disorders which facilitates the individual layers of skin for return to a normal rate of growth.

Still another principal advantage of the present invention is the provision of a method for treatment of skin disorders which minimizes complications encountered by an individual exposed to topical and/or internal medication received during treatment of a skin disease.

Still another principal advantage of the present invention is the provision of a method for treatment of skin disorders which minimizes relapse or recurrence of the skin disease following completion of a treatment regime.

Still another principal advantage of the present invention is the provision of a method for treatment of skin disorders which naturally returns moisture to areas of skin affected with the undesirable skin disease.

Still another principal advantage of the present invention is the provision of a method for treatment of skin disorders which includes a topical ointment having lard.

Still another principal advantage of the present invention is the provision of a method for treatment of skin disorders which includes a topical ointment having marigold flowers.

Still another principal advantage of the present invention is the provision of a method for treatment of skin disorders which includes a topical ointment having incense.

Still another principal advantage of the present invention is the provision of a method for treatment of skin disorders which includes a topical ointment having natural pure bee's wax.

Still another principal advantage of the present invention is the provision of a method for treatment of skin disorders which includes a topical ointment having rosemary flower.

Still another principal advantage of the present invention is the provision of a method for treatment of skin disorders which includes a topical ointment having nettles extract.

Still another principal advantage of the present invention is the provision of a method for treatment of skin disorders which includes a topical ointment having St. Johns wart oil.

Still another principal advantage of the present invention is the provision of a method for treatment of skin disorders which includes a topical treatment having marigold oil.

Still another principal advantage of the present invention is the provision of a method for treatment of skin disorders which includes the consumption of nettles tea.

Still another principal advantage of the present invention is the provision of a method for treatment of skin disorders where the lard, marigold flowers, pure bee's wax, incense, and rosemary flower are boiled for a period of time to not exceed 10 minutes, are removed from heat, are covered and are left for a period of 8 to 10 hours.

Still another principal advantage of the present invention is the provision of a method for treatment of skin disorders where the previously boiled mixture following cooling for 8 to 10 hours is melted and drained and where the nettles extract, the St. John's wart oil, and the marigold oil are added to provide a cream.

Still another principal advantage of the present invention is the provision of a method for treatment of skin disorders where the cooled cream is used as a natural topical ointment for the treatment of skin disorders by application into affected areas of skin at least once daily and preferably twice daily until such time as the affected skin has healed.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

The method for treatment of skin disorders such as dry skin, eczema, itchy skin, red skin, itchy eczema, inflamed skin, and/or cracked skin is disclosed herein. In general, the method for treatment of skin disorders involves the application of a topical ointment to an affected area of an individuals skin at least once per day and preferably twice per day for treatment of the undesirable skin condition.

The formulation of the ointment initiates with the acquisition of lard. The lard should be rinsed approximately 9 times in cold water prior to use in the formulation of the ointment described herein. Following rinsing, the lard should be drained. Approximately 0.5 kilograms of lard should be obtained and rinsed.

An individual should next obtain 2.5 ounces of full marigold flowers for combination with the lard as described above.

It should be noted that other portions of lard and full marigold flowers may be used at the discretion of an individual provided that the ratio of the proportions is maintained for the formulation of the ointment described herein.

An individual will next obtain approximately 100 grams of bee's wax for combination to the lard and marigold flowers. The bee's wax is preferably pure.

An individual will next obtain at least 10 pieces of incense where each piece of incense is approximately the size of a kernal of corn for addition to the pure bee's wax, marigold flowers and lard. More than 10 pieces of incense may be used at the discretion of an individual. The size of the incense may exceed the average size of a kernel of corn and relatively large portions of incense may be used as a portion of the treatment formulation.

An individual will next obtain at least three strips of rosemary flower for addition to the pure bee's wax, marigold flowers, lard, and incense. Again, it should be noted that the amount of the individual ingredients may vary at the discretion of an individual provided that a ratio of proportions of the identified ingredients is maintained.

The ingredients of the lard, marigold flowers, pure bee's wax, incense, and rosemary flower are then heated to boil and boiled for a period of approximately 5 to 10 minutes. Preferably the ingredients are heated within a clean porcelain and/or stainless steel utensil and are not heated within an aluminum utensil. The use of aluminum utensils may facilitate the burning of the mixture which is undesirable.

During the initial heating of the lard, full marigold flowers, pure bee's wax, incense, and strings of rosemary flower, the mixture should be stirred every 30 seconds to 1 minute to reduce risk of burning during the initial heating step.

The stirring and heating of the mixture will result in the provision of a liquid mixture having a pale gold color. After the heat has been applied to the liquid mixture following boiling for a period of 5 to 10 minutes, the heat should be terminated whereupon the mixture should be covered and allowed to stand for 8 to 10 hours. The mixture should be brought to full boil within the 5 to 10 minute period of time whereupon heat may be terminated for covering for 8 to 10 hours.

Following a period of time of 8 to 10 hours, heat may be reapplied to melt the mixture. At such time as the mixture becomes melted, the mixture may be drained whereupon two ounces of nettles extract should be added to the mixture in conjunction with two ounces of St. Johns wart oil and two ounces of marigold oil. The mixture including the two ounces of nettles extract and the two ounces of St. Johns wart oil and two ounces of marigold oil should then be mixed whereupon draining may occur. The draining of the mixture should occur through a clean fine tea filter which has not been previously utilized. The cream obtained following draining should be placed within new and clean containers which have previously been sterilized. A preferred type of container maybe glass jars and/or plastic jars. Following placement of the cream ointment within the containers, a suitable device may be used to seal the containers to prevent future contamination.

The containers of the ointment may be stored at room temperature or refrigerated at the discretion of an individual. In the preferred embodiment no preservatives are added to the ointment to facilitate storage.

The ointment may be then extracted from the containers for application to an affected area of skin for the treatment of the undesirable skin condition. Preferably the affected area of skin is treated twice daily, once in the morning and once during the evening to facilitate healing. During use, the ointment is preferably rubbed and/or massaged into an affected area of skin. Itching is normally terminated within two to three weeks and normally 10 days following the initiation of treatment of the ointment disclosed herein. The topical ointment should be applied to the affected area of skin twice daily until the undesirable skin condition has been cured.

The affected areas of skin treated with the ointment should not be exposed to soap, chemicals, or shampoo during the course of treatment. An individual may use a sensitive bar soap on non-affected areas of the body. The ointment applied to the affected areas of skin should be left on the skin all day and all night until such time as healing is achieved.

An individual using the ointment as described herein should avoid the consumption of caffeine and/or alcohol during treatment of an undesirable skin condition such as dry skin, eczema, itchy skin, red skin, itchy eczema, inflamed skin, and/or cracked skin.

In addition to the use of the ointment as described herein, an individual may also facilitate treatment of the undesirable skin condition by drinking two cups of nettles tea where one cup is consumed in the morning and one cup is consumed at night each and every day during the course of treatment.

The duration of treatment is generally dependent upon the length of time an individual has been inflicted with an undesirable skin condition. The treatment may be accomplished in a relatively short period of time of two to three weeks. Alternatively, in circumstances where an individual has previously suffered from an undesirable skin condition for 20 to 30 years, then treatment may be required to extend over a two or three month period of time.

During treatment, healing skin treated with the natural ointment described herein will initially assume a light brown color. As additional healing occurs, the color of the healing skin will change and clear to the normal skin color for the individual.

The above Examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below (e.g. claim 3 may be taken as alternatively dependent from claim 2; claim 4 may be taken as alternatively dependent on claim 2, or on claim 3; claim 6 may be taken as alternatively dependent from claim 5; etc.).

This completes the description of the preferred and alternate embodiments of the invention. Those skilled in the art may recognize other equivalents to the specific embodiment described herein which equivalents are intended to be encompassed by the claims attached hereto.

1. I claim a composition for the topical treatment of skin comprising:
a) lard;
b) marigold flowers mixed with said lard;
c) bee's wax mixed with said lard and said marigold flowers;
d) incense mixed with said lard, said marigold flowers, and said bee's wax; and
e) rosemary flower mixed with said lard, said marigold flowers, said bee's wax, and said incense.

2. The composition according to claim 1, further comprising nettles extract.

3. The composition according to claim 2, further comprising St. Johns wart oil.

4. The composition according to claim 2, further comprising marigold oil.

5. The composition according to claim 1, wherein said lard, said marigold flowers, said bee's wax, said incense, and rosemary flower are heated.

6. The composition according to claim 4, wherein said lard, said marigold flowers, said bee's wax, said incense, said rosemary flower, said nettles extract, said St. Johns wart oil, and said marigold oil are mixed to provide a cream adapted for use as a topical treatment of said skin.

7. The composition according to claim 6, said lard comprising 0.5 kilograms.

8. The composition according to claim 7, said marigold flowers comprising 2.5 ounces.

9. The composition according to claim 8, said bee's wax comprising 100 grams.

10. The composition according to claim 9, said rosemary flower comprising 3 strings of rosemary flower.

11. The composition according to claim 10, said nettles extract comprising 2 ounces.

12. The composition according to claim 11, said St. Johns wart oil comprising 2 ounces.

13. The composition according to claim 11, said marigold oil comprising 2 ounces.

14. I claim a method for formulation of a topical treatment of skin comprising:
a) mixing approximately 0.5 kilograms of lard with approximately 2.5 ounces of marigold flowers;
b) adding approximately 100 grams of bee's wax to said mixed lard and marigold flowers;
c) adding incense to said mixed bee's wax, said mixed marigold flowers, and said lard;
d) adding rosemary flower to said mixed incense, said bee's wax, said marigold flowers, and said lard;
e) heating said mixed lard, said marigold flowers, said bee's wax, said incense, and said rosemary flower to boil for approximately 5 to 10 minutes;
f) cooling said heated lard, said marigold flowers, said bee's wax, said incense, and said rosemary flower by removal from heat and covering for a period of time of at least 8 hours;
g) reheating said lard, said marigold flowers, said bee's wax, said incense, and said rosemary flower;
h) adding nettles extract to said reheated lard, said marigold flowers, said bee's wax, said incense, and said rosemary flower; and
i) adding St. Johns wart oil and marigold oil to said nettles extract, said rosemary flower, said bee's wax, said marigold flowers, and said lard, draining said mixed lard, said marigold flowers, said bee's wax, said incense, said rosemary flower, said nettles extract, said St. Johns wart oil, and said marigold oil to provide a cream.

15. I claim a method for treatment of skin comprising:
a) forming a topical treatment for said skin, said topical treatment comprising:
i) lard;
ii) marigold flowers;
iii) bee's wax;
iv) incense;
v) rosemary flower;
vi) nettles extract;
vii) marigold oil;
viii) St. Johns wart oil, wherein said lard, said marigold flowers, said bees wax, said incense, said rosemary flower, said nettles extract, said marigold oil, and said St. Johns wart oil are mixed and heated for formulation of an ointment; and
b) applying said ointment to said skin at least once per day to treat said skin.

16. The method according to claim 15, said lard comprising 0.5 kilograms.

17. The method according to claim 16, said marigold flowers comprising 2.5 ounces.

18. The method according to claim 17, said bee's wax comprising 100 grams.

19. The method according to claim 18, said rosemary flower comprising 3 strings.

20. The method according to claim 19, said nettles extract comprising 2 ounces.

21. The method according to claim 20, said St. Johns wart oil comprising 2 ounces.

22. The method according to claim 21, said marigold oil comprising 2 ounces.

23. The method according to claim 22, said method comprising the consumption of two cups of nettles tea per day during use of said ointment.
